(19) United States
(12) Patent Application Publication
(10) Pub. No.: US 2016/0151486 A1
(43) Pub. Date: Jun. 2, 2016

(54) CD40 Signalling Inhibitor and a Further Compound, Wherein the Further Compound is a Bile Acid, a Bile Acid Derivative, an TGR5-Receptor Agonist, an FXR Agonist or a Combination Thereof, for the Treatment of Chronic Inflammation, and the Prevention of Gastrointestinal Cancer or Fibrosis

(71) Applicant: Fast Forward Pharmaceuticals B.V., Utrecht (NL)

(72) Inventors: Anton Egbert Peter Adang, Utrecht (NL); Mark de Boer, Utrecht (NL); Marielle Marie Guillaume Louis Thewissen, Utrecht (NL)

(73) Assignee: Fast Foward Pharmaceuticals B.V., Utrecht (NL)

(21) Appl. No.: 14/897,295

(22) PCT Filed: Jun. 13, 2014
(86) PCT No.: PCT/NL2014/050390

§ 371 Date: Dec. 10, 2015

(30) Foreign Application Priority Data
Jun. 13, 2013 (EP) 13171913.0

(51) Int. Cl. A61K 39/395; A61K 45/06; C07K 16/28

(52) U.S. Cl. CPC A61K 39/3955; C07K 16/2875; A61K 45/06; C07K 2317/76

(57) ABSTRACT

The invention provides a CD40 signalling inhibitor and a further compound for use in the treatment of chronic inflammatory disease in an individual in need thereof, wherein the further compound is a bile acid, a bile acid derivative, an TGR5-receptor agonist, an FXR-receptor agonist or a combination thereof. Also provided is a CD40 signalling inhibitor and a further compound for use in the prevention of cancer and/or fibrosis, wherein the further compound is a bile acid, a bile acid derivative, an TGR5-receptor agonist, an FXR-receptor agonist or a combination thereof.

The invention relates to the field of medicaments. The invention in particular relates to means and methods for treating individuals that suffer or are at risk of suffering from a chronic inflammation. The invention also relates to means and methods for the prevention of cancer and fibrosis. More specifically the invention relates to CD40 signalling inhibitors, such as CD40 binding antibodies, that inhibit activation of the CD40 receptor and bile acid or bile acid derivatives thereof for use in the treatment of chronic inflammatory or autoimmune diseases with an inflammatory component or for the prevention of gastrointestinal cancer or fibrosis, preferably liver, kidney or gastrointestinal fibrosis.

Inflammation, the response of tissue to injury, is characterized in the acute phase by increased blood flow and vascular permeability along with the accumulation of fluid, leukocytes, and inflammatory mediators such as cytokines. In the subacute/chronic phase (hereafter referred to as the chronic phase), it is characterized by the development of specific humoral and cellular immune responses to the pathogen(s) present at the site of tissue injury. During both acute and chronic inflammatory processes, a variety of soluble factors are involved in leukocyte recruitment through increased expression of cellular adhesion molecules and chemoattraction. Many of these soluble mediators regulate the activation of the resident cells (such as fibroblasts, endothelial cells, tissue macrophages, and mast cells) and the newly recruited inflammatory cells (such as monocytes, lymphocytes, neutrophils, and eosinophils), and some of these mediators result in the systemic effects of the inflammatory process (e.g. fever, hypotension, synthesis of acute phase proteins, leukocytosis, cachexia) (C. A. Feghali et al., 1997, Frontiers in Bioscience 2, pp 12-26) Apart from soluble factors there are also cell-cell mediated signalling pathways, including the CD40 signalling pathway, that are relevant for the maintenance and severity of the chronic inflammation. Most of the involved soluble and cell-associated factors have pleiotropic effects.

Inflammatory responses can be triggered by components of microbes as well as by macromolecules, such as proteins and polysaccharides, and small chemicals that are recognized as foreign. Inflammatory responses and mechanisms are intended to protect individuals from infection and eliminate foreign substances but are also capable of causing tissue injury and disease in some situations. Under some conditions, even self (autologous) molecules can elicit an inflammatory response, such reactions are called autoimmune responses and diseases caused by these reactions are collectively called autoimmune diseases (Abbas et al, Cellular and Molecular Immunology 7E).

In the present invention it was found that a CD40 signalling inhibitor and further compound, can be favourable combined in the treatment of chronic inflammatory disease and in the prevention of cancer or fibrosis. The further compound is preferably a bile acid, a bile acid derivative, an TGR5-receptor agonist, an FXR-receptor agonist or a combination thereof. The present invention now provides a CD40 signalling inhibitor and a further compound for use in the treatment of chronic inflammatory disease in an individual in need thereof, wherein the further compound is a bile acid, a bile acid derivative, an TGR5-receptor agonist, an FXR-receptor agonist or a combination thereof. The invention also provides a CD40 signalling inhibitor and a further compound for use in the prevention of cancer and/or fibrosis, wherein the further compound is a bile acid, a bile acid derivative, a TGR5-receptor agonist, an FXR-receptor agonist or a combination thereof. In a preferred embodiment said fibrosis is fibrosis of the liver, kidney or gastrointestinal fibrosis.

Bile acids and bile acid derivatives mediate a plethora of different effects besides their main function which is to facilitate the formation of micelles, which promotes processing of dietary fat. These auxiliary effects include anti-inflammatory effects. These and other effects are thought to be mediated among others by binding of the bile acid or derivative to specific bile acid receptors. Preferred examples of bile acid receptors are the receptors TGR5 and FXR. TGR5 is a G-protein coupled receptor also known as a G protein-coupled bile acid receptor 1 (GPBAR1), G-protein coupled receptor 19 (GPCR19), membrane-type receptor for bile acids (M-BAR). TGR5 is a protein that in humans is encoded by the GPBAR1 gene. TGR5 is encoded by a single exon that maps to chromosome 1C3 in mouse and 2q35 in humans. TGR5 is ubiquitously expressed, but its expression levels vary among different tissues, with high expression in liver, intestine, brown adipose tissue, and spleen. Different bile acids act differently on TGR5 and FXR activation, indicating that these two receptors have differential functions in mediating the effects of bile acids (Xiaosong Chen et al (2011) Exp. Diabetes Res. Vol 2011: pp 1-5). The farnesoid X receptor (FXR) also known as the bile acid receptor or BAR (gene symbol NR1H4) is a member of the nuclear receptor family. FXR functions as the chief sensor of intracellular levels of bile acids (the end products of cholesterol catabolism) and is the main executor of bile acid-induced transcriptional programmes. Bile acids directly interact with the ligand-binding domain of FXR and enhance or antagonize the transactivation function of FXR. In accordance with its function as the bile acid receptor, FXR is most abundantly expressed in the tissues commonly exposed to bile acids in normal physiology: liver, intestine, and kidneys. Signalling via FXR and TGR5 modulates several metabolic pathways, regulating not only BA synthesis and enterohepatic recirculation, but also triglyceride, cholesterol, glucose and energy homeostasis (reviewed in Fiorucci et al (2009) Trends Pharmacol Sci. Vol 30:p 570-580).

Bile acids have long been known to exert direct regulatory function on cells of innate immunity. An example of such a bile acids is chenodeoxycholic acid (CDCA), a primary bile acid and FXR ligand. CDCA negatively regulates IL1b, IL6 and TNF release from LPS-primed macrophages (Calmus 1992). FXR activation was shown to antagonize NFκB activity and thereby antagonize pro-inflammatory gene expression (Wang 2008, Vavassori 2009).

Preferred TGR5 agonists for use in the invention are described among other in Hiroyuki Sato et al (2008). J. Med. Chem. 51, 1831-1841. Sato et al have recently reported that 23-alkyl-substituted and 6,23-alkyl-disubstituted derivatives of chenodeoxycholic acid, such as the 6R-ethyl-23(S)-methylchenodeoxycholic acid, are potent and selective agonists of TGR5, in particular, it was shown that methylation at the C23-(S) position of natural bile acids confers a marked selectivity to TGR5 over FXR activation, whereas the 6R-alkyl substitution increases the potency at both receptors. The screening of libraries of nonsteroidal compounds and natural products has led to the disclosure of 6-methyl-2-oxo-4-thiophen-2-yl-1,2,3,4-tetrahydropyrimidine-5-carboxylic acid benzyl ester (WO2004067008) and oleanoic acid as structurally diverse TGR5 agonists. Recently various bile acid and bile acid derivatives have been synthesized. For instance, enantiomeric chenodeoxycholic acid (CDCA) and lithocholic acid (LCA). Sato et al (2008) describe various other TGR5 agonists and is incorporated by reference herein for reference to TGR5 agonists. The paper describes both TGR5 selective agonists and TGR5, FXR duo agonists. A TGR5 agonist and an FXR agonist have various properties. For the present invention a compound is an TGR5 agonist if it is active in the TGR5 agonist assay described in Sato et al (2008). For the present invention a compound is an FXR agonist if it is active in the FXR agonist assay described in Sato et al (2008). A compound is a duo TGR5, FXR agonist if it is active in the TGR5 and FXR assays described in Sato et al (2008). This reference is therefor also enclosed by reference herein for a description of the TGR5 and the FXR agonist tests. Preferred TGR5 agonists of the invention are described in (Gioiello et al (2012) Expert Opin. Ther. Patents. Vol 22: pp 1399-1414). Particularly preferred is a TGR5 agonist as depicted in table 1, table 2, table 3, FIG. 1, FIG. 2 or FIG. 3 of (Gioiello et al (2012) Expert Opin. Ther. Patents. Vol 22: pp 1399-1414). Preferred is also a TGR5 agonist as depicted in table 1, table 2, FIG. 2, FIG. 3, FIG. 4 or FIG. 5 of the present application. A preferred TGR5 agonist is UDCA (FIG. 5). Preferred is also a TGR5 agonist as described in WO2008/002573; WO2008/091540; WO2010/059859, WO2010/059853 or WO2010/014836. A preferred FXR agonist is an agonist as described in WO2010/059853; WO2007/095174; WO2008/002573 or WO2002/072598. A preferred FXR agonist is an agonist of FIG. 3 of ref Modica S. Deciphering the nuclear bile acid receptor FXR paradigm. NRS 2010; 8:pp 1-28. A preferred FXR agonist is an FXR agonist of FIG. 6.

TGR5 agonists are also described in US2012/0115832. This reference is therefore also incorporated by reference herein, particularly for the description of the various TGR5 agonists.

FXR agonists are also described in WO2005/082925 and in US2008/0182832. These references are therefore also incorporated by reference herein, particularly for the description of the various FXR, agonists. A preferred FXR agonist is an agonist as described in WO2010/059853; WO2007/095174; or WO2002/072598.

The CD40 molecule is a 50 kDa type I membrane glycoprotein and is expressed on B cells, monocytes/macrophages, dendritic cells (DCs) and activated endothelial cells.¹⁻⁶ Under certain conditions, CD40 can also be found on fibroblasts, epithelial cells, keratinocytes and other cells.⁷ CD40 ligand (CD40L, CD154), a 32 kDa type II integral membrane glycoprotein, is transiently expressed on activated CD4⁺ T cells and a small population of activated CD8⁺ T cells.^{8, 9} In addition, CD40L has been found on a number of other cell types after activation, including mast cells, basophils, B cells, eosinophils, DCs and platelets.^{10, 11} The CD40 pathway is considered a key switch in both the initiation and effector stage of inflammatory responses.

Binding of CD40 and CD40L (also referred to as ligation of CD40 and CD40L) initiates a signalling cascade inside the CD40 expressing cell. Signalling by CD40 is typically inhibited by means of an antibody that binds CD40 or CD40L. The CD40-CD40L interaction can be inhibited with monoclonal antibodies (Mabs) against either CD40L or CD40. The expression of CD40L on activated platelets has resulted in thrombo-embolic events during treatment of humans with IgG1 anti-human CD40L Mabs at higher dose levels and termination of the development of these Mabs^{12, 13}. Inhibiting CD40 signalling via a CD40 binding antibody therefore seems a more attractive approach, in humans. The inhibitory activity of Mab 5D12 (anti-human CD40) was demonstrated in various in vitro studies using different CD40-bearing cell types^{14, 15} and chimeric 5D12 (ch5D12) CD40 inhibitory activity was validated in vivo using various non-human primate disease models′^{16, 17}. ch5D12 is a molecularly engineered human IgG4 antibody containing the murine variable domains of the heavy and light chains of 5D12 and was constructed to reduce the potential for immunogenicity and to enhance the in vivo half-life of the murine 5D12 Mab when used in humans.

Like many receptors, the CD40 receptor does not signal in the absence of CD40L or equivalent. A CD40 signalling inhibitor does therefore not inhibit signalling by the receptor when there is no activator. Thus a CD40 signalling inhibitor inhibits signalling under conditions that the CD40 receptor would otherwise be active (i.e. in the absence of the inhibitor). The physiological way of activating CD40 signalling is by providing the CD40 expressing cell with CD40L. This can be done by providing a CD40L expressing cell, or by providing soluble CD40L. A compound is a CD40 signalling inhibitor when it reduces the activation of CD40 signalling in CD40 expressing cells by 50% or more. In such tests the compound is preferably added before the compound or cell is added that activates the CD40 receptor. However, this need not always be the case. The compound that activates the CD40 receptor in this assay is preferably CD40L, either by providing a CD40L expressing cell, or preferably, by providing soluble CD40L. Presently various CD40 binding antibodies are available that can activate signalling of the CD40 receptor upon binding. Such antibodies are also referred to as CD40 agonists.

A CD40 signalling inhibitor can be a CD40 binding molecule, a CD40L binding molecule or a combination thereof. The CD40 or CD40L binding molecule is typically an antibody or fragment or derivative or mimic thereof. Various antibody CD40 signalling inhibitors are known in the art. A preferred CD40 signalling inhibitor is a CD40 binding antibody that inhibits CD40 signalling in CD40 expressing cells by 50% or more, preferably in a test as described herein before. In a preferred embodiment the CD40 signalling inhibitor is a CD40 binding CD40 inhibitor. In a preferred embodiment the CD40 signalling inhibitor is a monoclonal antibody or an antigen-binding portion thereof that binds to and inhibits activation of human CD40. The antibody preferably comprises a variable domain amino acid sequence selected from the group consisting of CD40 binding antibodies 5D12, ch5D12 but also PG102, and ASKP1240 (EP1391464). A further antibody preferably comprises a variable domain amino acid sequence selected from the group consisting of CD40 binding antibodies of US2011/0243932. Non-limiting but preferred examples are the aforementioned CD40 binding antibodies 5D12, ch5D12 but also PG102, US2011/0243932 and ASKP1240. PG102 and other CD40 signalling inhibiting CD40 binding antibodies are described in WO2007/129895 Such antibodies bind CD40 and inhibit CD40 receptor signalling in a test as described earlier by at least 50%. Particularly preferred CD40 signalling inhibitors are ch5D12, PG102, HCD122 (CHIR-12.12, lucatumumab), US2011/0243932 and ASKP1240 (EP1391464). In a particularly preferred embodiment the CD40 signalling inhibitor is PG102 (the amino acid sequence of the variable regions is depicted in FIG. 1). Various CD40 antibodies have been tested in clinical trials (A phase 1 study of lucatumumab, a fully human anti-CD40 antagonist monoclonal antibody administered intravenously to patients with relapsed or refractory multiple myeloma William Bensinger, et al., British Journal of Haematology, Vol 159, Issue 1, pages 58-66, October 2012 and a phase I study of the anti-CD40 humanized monoclonal antibody lucatumumab (HCD122) in relapsed chronic lymphocytic leukemia. Leuk Lymphoma. 2012 November; 53(11):2136-42, 2012 Jun. 12).

Other CD40 signalling inhibitors bind CD40L. These inhibitors typically prevent binding of CD40L to CD40. Such CD40L binding inhibitor is preferably a CD40L binding antibody or fragment or derivative or mimic thereof. A preferred CD40L binding antibodies that are CD40 signalling inhibitors are MR-1, IDEC131) (E6040⁰, IDEC hu5C8 (BG9588) described in Vincenti (2002), Am. J. of Transplantation Vol 2, pp 898-903 and references therein. The IDEC molecules are against human CD40L whereas MR-1 is against mouse CD40L.

Presently there are many different proteins with similar binding properties in kind as antibodies. These molecules are further referred to as an antibody equivalent or antibody part or derivative or mimic. In the context of the present invention such antibody equivalents and parts and mimics and derivatives are considered to be equivalent to the antibody as provided in the means, uses and methods of the invention. Non-limiting examples of such antibody equivalents are non-antibody scaffold protein binders such as, but not limited to, anticalins, C-type lectin domain binders, avimers, Adnectins, and DARPins (Designed Ankyrin Repeat Proteins) (ref. Sheridan C. Nature Biotechnology 2007, (25), 365-366.)

A non-limiting example of an antibody part or derivative contains a variable domain of a heavy chain and/or a light chain of an antibody or an equivalent thereof. Non-limiting examples of such proteins are VHH, nanobodies, Human Domain Antibodies (dAbs), Unibody, Shark Antigen Reactive Proteins (ShArps), Small Modular ImmunoPharmaceutical (SMIP™) Drugs, monobodies and/or IMabs (ref. Sheridan C. Nature Biotechnology 2007, (25), 365-366.). Preferred antibody parts or derivatives have at least a variable domain of a heavy chain and a light chain of an antibody or equivalents thereof. Non-limiting examples of such binding molecules are F(ab)-fragments and Single chain Fv fragments. Many different proteins exist that have an IG-fold that can be manipulated to specifically bind a target. Such manipulated proteins are considered equivalents or mimics of an antibody. In a preferred embodiment the CD40 or CD40L binding molecule is an antibody. The antibody may be a natural antibody or a synthetic antibody. In a preferred embodiment an antibody comprises the CDR1, CDR2, CDR3 regions of an antibody. However, artificial generation of CDR like regions such as can be selected for instance via phage display are also included in the present invention. In a preferred embodiment said antibody is a human, humanized or human-like antibody. Particularly preferred are binding molecules that (apart from their specificity) do not further interact with the immune system. In case of antibodies it is preferred that said antibody comprises an IgG4 constant region, or an IgG4 like constant region. For instance it is possible to mutate the constant region of an IgG1 molecule such that it no longer activates the complement system upon binding to its target.

The antibodies used in the present invention may be from any animal origin including birds and mammals (e.g., human, murine, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries (including, but not limited to, synthetic libraries of immunoglobulin sequences homologous to human immunoglobulin sequences) or from mice that express antibodies from human genes. For some uses, including in vivo therapeutic or diagnostic use of antibodies in humans and in vitro detection assays, it may be preferred to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences or synthetic sequences homologous to human immunoglobulin sequences. See also U.S. Pat. Nos. 4,444,887 and 4,716,111; and PCT publications WO 98/46645, WO 98/50433, WO 98/24893 and WO98/16654, each of which is incorporated herein by reference in its entirety. The antibodies to be used with the methods of the invention include derivatives that are modified, i.e, by the covalent attachment of any type of molecule to the antibody such that covalent attachment. Additionally, the derivative may contain one or more non-classical amino acids. In certain embodiments of the invention, the antibodies to be used with the invention have extended half-lives in a mammal, preferably a human, when compared to unmodified antibodies. Antibodies or antigen-binding fragments thereof having increased in vivo half-lives can be generated by techniques known to those of skill in the art (see, e.g., PCT Publication No. WO 97/34631). In certain embodiments, antibodies to be used with the methods of the invention are single-chain antibodies. The design and construction of a single-chain antibody is well known in the art. In certain embodiments, the antibodies to be used with the invention bind to an intracellular epitope, i.e., are intrabodies. An intrabody comprises at least a portion of an antibody that is capable of immuno-specific binding an antigen and preferably does not contain sequences coding for its secretion. Such antibodies will bind its antigen intracellular. In one embodiment, the intrabody comprises a single-chain Fv (“sFv”). In a further embodiment, the intrabody preferably does not encode an operable secretory sequence and thus remains within the cell. Generation of intrabodies is well-known to the skilled artisan and is described for example in U.S. Pat. Nos. 6,004,940; 6,072,036; 5,965,371, which are incorporated by reference in their entireties herein. In one embodiment, intrabodies are expressed in the cytoplasm. In other embodiments, the intrabodies are localized to various intracellular locations. In such embodiments, specific localization sequences can be attached to the intranucleotide polypeptide to direct the intrabody to a specific location. The antibodies to be used with the methods of the invention or fragments thereof can be produced by any method known in the art for the synthesis of antibodies, in particular, by chemical synthesis or preferably, by recombinant expression techniques. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art. Examples of phage display methods that can be used to make the antibodies of the present invention include those disclosed in WO97/13844; and U.S. Pat. Nos. 5,580,717, 5,821,047, 5,571,698, 5,780,225, and 5,969,108; each of which is incorporated herein by reference in its entirety. As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324. It is also possible to produce therapeutically useful IgG, IgA, IgM and IgE antibodies. For a detailed discussion of the technology for producing human antibodies and human monoclonal antibodies and protocols for producing such antibodies, see, e.g., PCT publication No. WO 98/24893. All references cited herein are incorporated by reference herein in their entirety. In addition, companies such as Medarex, Inc. (Princeton, N.J.), Abgenix, Inc. (Freemont, Calif.) and Genpharm (San Jose, Calif.) can be engaged to provide human antibodies directed against a selected antigen using technology similar to that described above. Recombinant expression used to produce the antibodies, derivatives or analogs thereof (e.g., a heavy or light chain of an antibody of the invention or a portion thereof or a single chain antibody of the invention), requires construction of an expression vector containing a polynucleotide that encodes the antibody and the expression of said vector in a suitable host cell or even in vivo. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy, the entire light chain, or both the entire heavy and light chains. The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below. A variety of host-expression vector systems may be utilized to express the antibody molecules as defined herein In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. Once an antibody molecule to be used with the methods of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification. As stated above, according to a further aspect, the invention provides an antibody or equivalent or derivative thereof, as defined above for use in therapy. For therapeutic treatment, antibodies, or equivalent or derivative thereof, may be produced in vitro and applied to the subject in need thereof. The antibody or equivalent or derivative thereof, may be administered to a subject by any suitable route, preferably in the form of a pharmaceutical composition adapted to such a route and in a dosage which is effective for the intended treatment. Therapeutically effective dosages of the antibodies required for decreasing the rate of progress of the disease or for eliminating the disease condition can easily be determined by the skilled person. Alternatively, antibodies may be produced by the subject itself by using in vivo antibody production methodologies as described above. Suitably, the vector used for such in vivo production is a viral vector, preferably a viral vector with a target cell selectivity for specific target cell referred to herein. Therefore, according to a still further aspect, the invention provides the use of an antibody or equivalent or derivative thereof, as defined above in the manufacture of a medicament for use in the treatment of a subject to achieve the said therapeutic effect. The treatment comprises the administration of the medicament in a dose sufficient to achieve the desired therapeutic effect. The treatment may comprise the repeated administration of the antibody. According to a still further aspect, the invention provides a method of treatment of a human comprising the administration of an antibody or equivalent or derivative thereof, as defined above in a dose sufficient to achieve the desired therapeutic effect.

The chronic inflammatory disease is preferably an autoimmune disease with an inflammatory component. The chronic inflammatory disease is preferably a chronic inflammatory disease of the liver, of the kidney, of the gastrointestinal tract, of the cardiovascular system or the metabolic system. A preferred chronic inflammatory disease of the liver is a vanishing bile duct syndrome (VBDS), primary biliary cirrhosis (PBC), bile acid diarrhea (chronic diarrhea), primary sclerosing cholangitis (PSC), autoimmune hepatitis, liver transplant associated graft versus host disease, portal hypertension, non-alcoholic steatohepatitis (NASH) or non-alcoholic fatty liver disease (NAFLD). A preferred said chronic inflammatory gastrointestinal disease is a chronic inflammation of the pancreas, Crohn's disease, or ulcerative colitis. A chronic inflammatory cardiovascular disease is atherosclerosis and a preferred chronic inflammatory disease of the metabolic system is obesity, insulin resistance, type I diabetes or type II diabetes.

The chronic inflammatory or autoimmune disease is preferably a chronic inflammatory or autoimmune disease of the liver, of the kidney, of the gastrointestinal tract, of the cardiovascular system or the metabolic system. A preferred chronic inflammatory or autoimmune disease of the liver is primary biliary cirrhosis (PBC), bile acid diarrhea (chronic diarrhea), primary sclerosing cholangitis (PSC), autoimmune hepatitis, liver transplant associated graft versus host disease, portal hypertension, non-alcoholic steatohepatitis (NASH) or non-alcoholic fatty liver disease (NAFLD). A preferred said chronic inflammatory or autoimmune gastrointestinal disease is a chronic inflammation of the pancreas, Crohn's disease, or ulcerative colitis. A chronic inflammatory cardiovascular disease is atherosclerosis and a preferred chronic inflammatory or autoimmune disease of the metabolic system is obesity, insulin resistance, metabolic syndrome, type I diabetes or type II diabetes.

Considering the chronic inflammation is often associated with serious diseases as cancer and fibrosis, and considering that the present invention at least ameliorates the chronic inflammation, the CD40 signalling inhibitor and further compound can also be used to at least delay the onset of cancer and to at least reduce the fibrosis in the treated individuals. At least when compared to the same or similar individuals that do not receive cancer prevention or fibrosis prevention treatments. Existing fibrosis is often not reversible. By preventing fibrosis is therefore meant preventing fibrosis that would otherwise have occurred, had the treatment not been given.

The bile acid or bile acid derivative is preferably a bile acid, or derivative as mentioned herein above. Presently it is possible to synthesize many of the bile acids and bile acid derivatives in vitro. Thus a bile acid derivative as used herein does not only refer to compounds that are derived from a bile acid, but also to the synthesized compounds with the same structure as the compounds derived from bile acid. A chenodeoxycholic acid derivative is a preferred bile acid derivative. Preferably the bile acid derivative is 6-alpha-ethyl chenodeoxycholic acid, or a 23-substituted bile acid.

A preferred bile acid is ursodeoxycholic acid or chenodeoxycholic acid.

It is preferred that the bile acid or bile acid derivative is an FXR and/or TGR5 signalling activator. Such compounds are in the art also referred to as FXR-agonists or TGR5-agonists. As mentioned earlier, various TGR5 signalling activators are also FXR signalling activators.

The invention further provides a method for the treatment of an individual suffering from a chronic inflammation, said method comprising administering to the individual in need thereof, an CD40 signalling inhibitor and a further compound, wherein the further compound is a bile acid, a bile acid derivative, an TGR5-receptor agonist, an FXR-receptor agonist or a combination thereof.

The invention also provides a kit comprising a CD40 signalling inhibitor and a further compound wherein the further compound is a bile acid, a bile acid derivative, a TGR5-receptor agonist, an FXR-receptor agonist or a combination thereof. The kit is preferably for use in the treatment of chronic inflammatory disease in an individual in need thereof or for use in the prevention of cancer and/or fibrosis in an individual in need thereof.

### BRIEF DESCRIPTION OF THE DRAWINGS

FIG. 1. Amino acid sequence of antibody PG102

FIG. 2. A. Various bile acid scaffold modifications. B. Bile acid derivatives, structure and strength. C. bile acid derivative of Novartis (from Gioiello et al (2012) Expert Opin. Ther. Patents. Vol 22: pp 1399-1414).

FIG. 3. Various TGR agonists. Structure and potency. (from Gioiello et al (2012) Expert Opin. Ther. Patents. Vol 22: pp 1399-1414).

FIG. 4. Various natural TGR5 agonists and potency. (from Gioiello et al (2012) Expert Opin. Ther. Patents. Vol 22: pp 1399-1414).

FIG. 5. TGR5 and FXR agonists.

FIG. 6. FXR agonists from. Modica S. Deciphering the nuclear bile acid receptor FXR paradigm. NRS 2010; 8:pp 1-28.

FIG. 7: Inhibition of cytokine secretion by PG102 and 6-ECDCA. PBMC were stimulated with A) megaCD40L or B) megaCD40L and LPS to induce cytokine secretion. 6-ECDCA and/or PG102 were added to the cultures and their effect on TNF, IL-6, IL-8, IL-1β and IL-12p70 levels in the culture supernatant was evaluated.

FIG. 8: Body weight of the mice during the experiment. Colitis was induced in mice by administration of 2.5% (wt/vol) DSS in drinking water from day 3 onwards for 8 days. Body weight was measured daily and expressed relative to the body weight at day 1.

FIG. 9: Intestinal permeability determined by the FITC concentration in the plasma 4 h after oral gavage. Mice were given FITC by oral gavage after 8-days of DSS-treatment. 4 h after FITC administration, mice were sacrificed and the amount of fluorescence in the blood was determined as a marker for intestinal permeability.

FIG. 10: Length of the colon. At the end of the experiment mice were sacrificed and the colon was isolated. The length of the colon was measured as a measure of colonic inflammation.

FIG. 11: Analysis of granulocytes in the spleen. At the end of the experiment mice were sacrificed and the spleen was isolated. Spleen cells were stained with antibodies to GR-1 and CD11b and the relative contribution of granulocytes was determined by FACS analysis.

FIG. 12: TNF release by spleen cells upon stimulation with PMA and Ionomycin. At the end of the experiment mice were sacrificed and the spleen was isolated. Spleen cells were stimulated with PMA and Ionomycin for 4.5 h and TNF release was measured by ELISA.

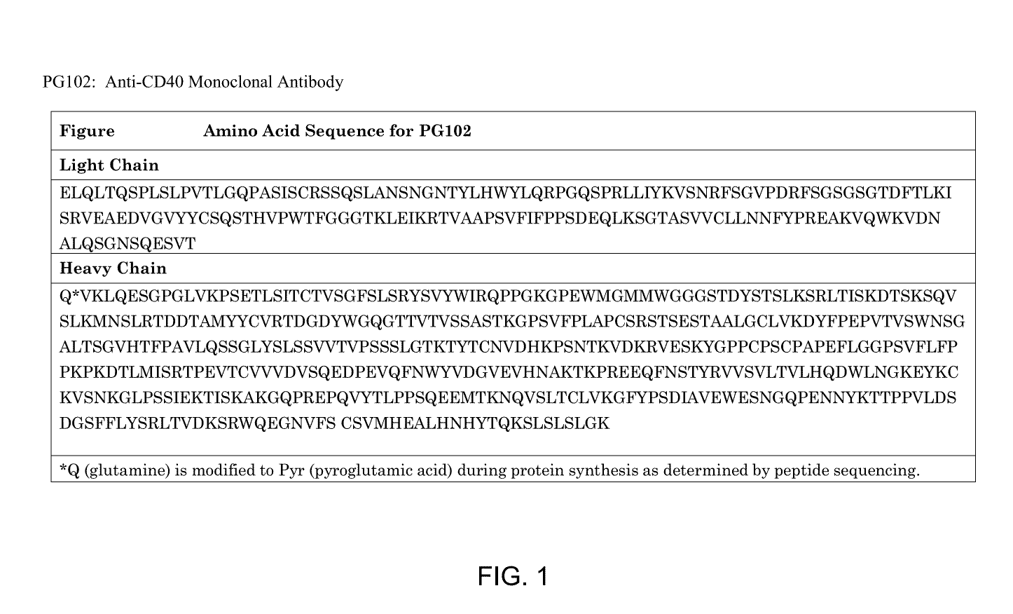

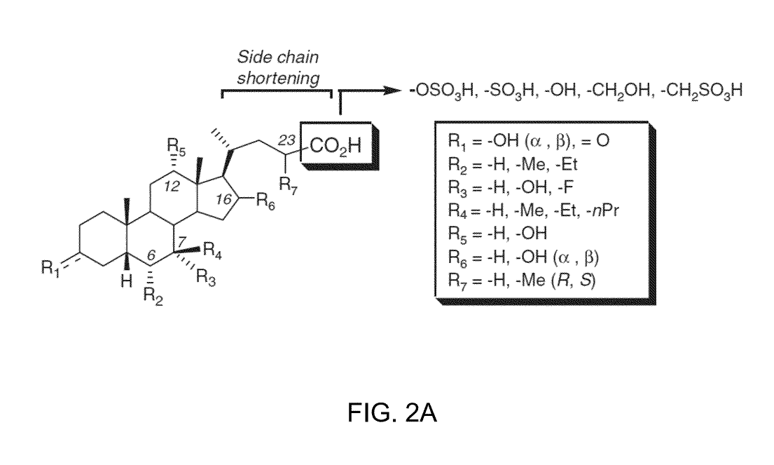

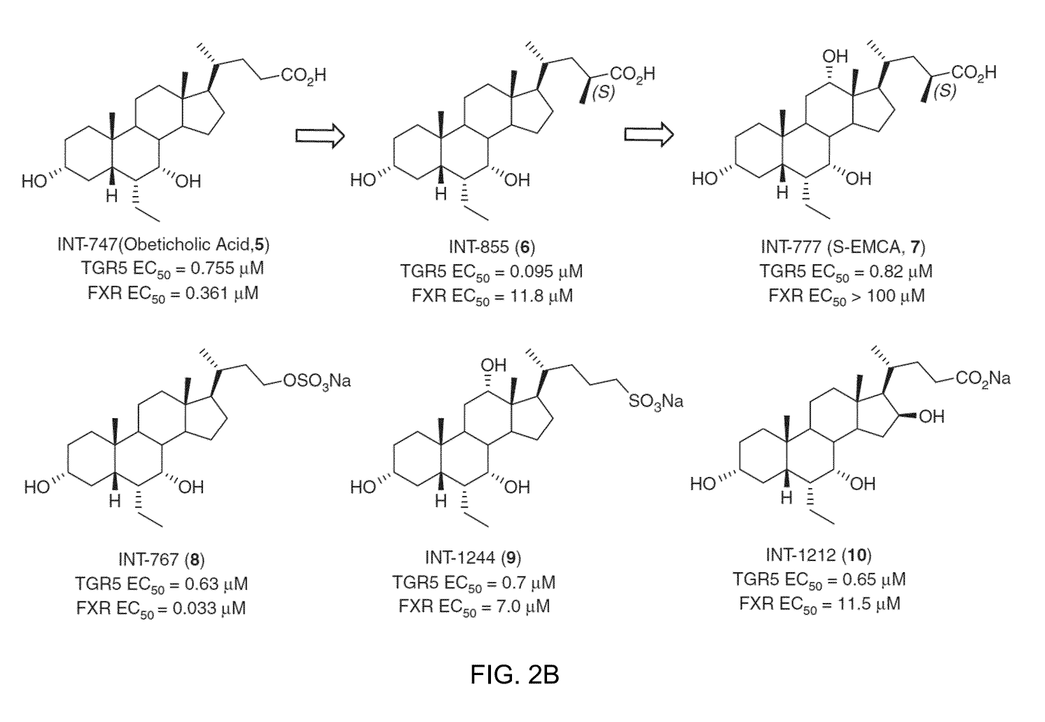

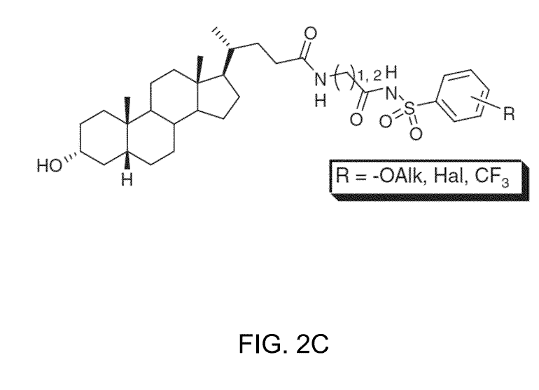

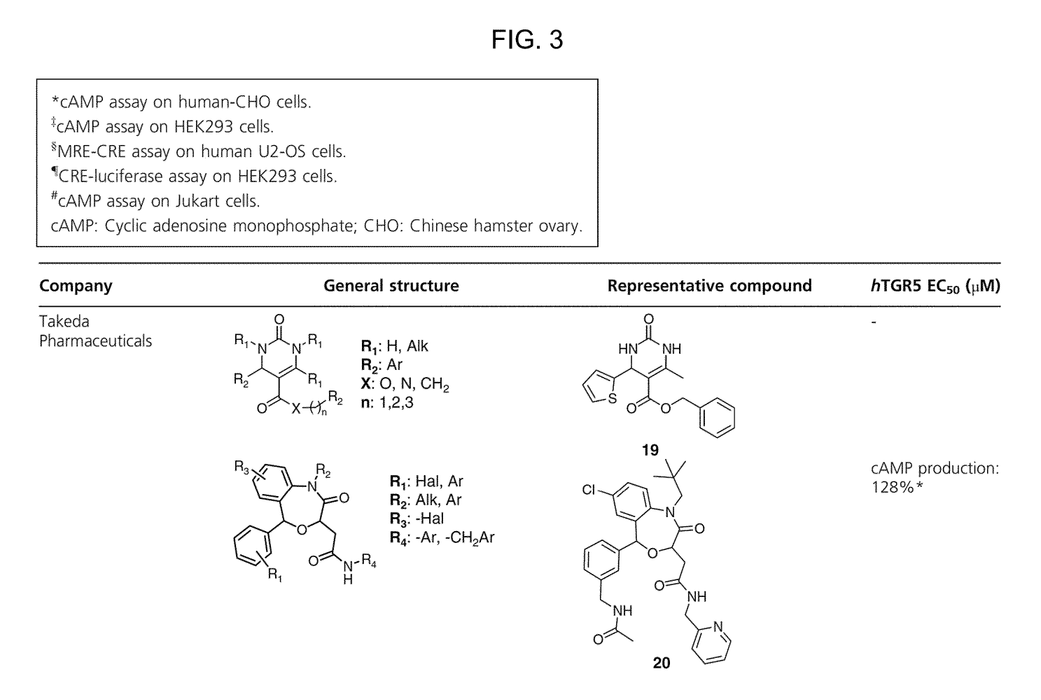

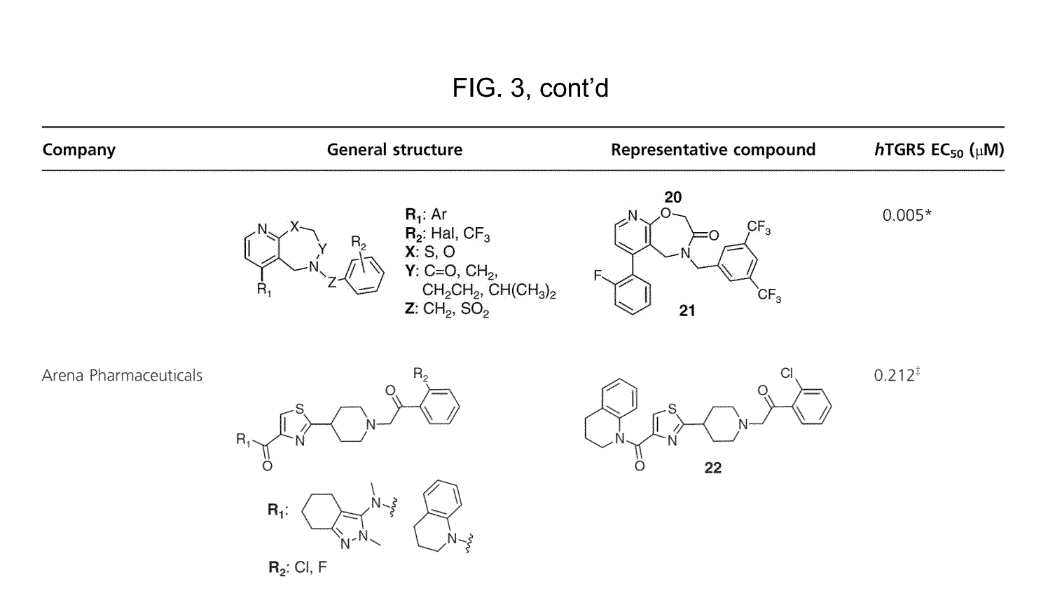

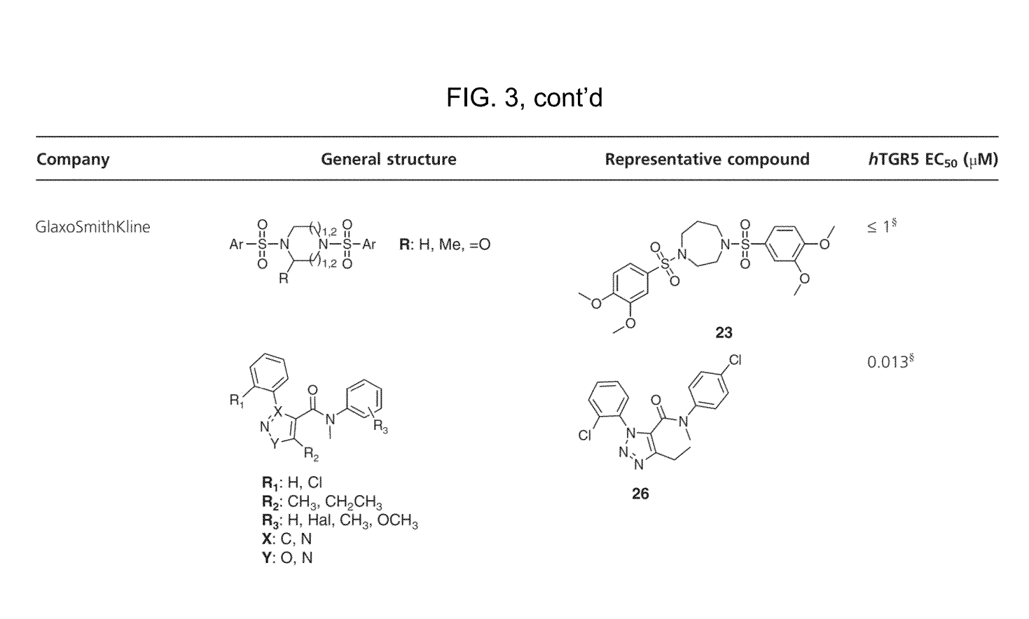

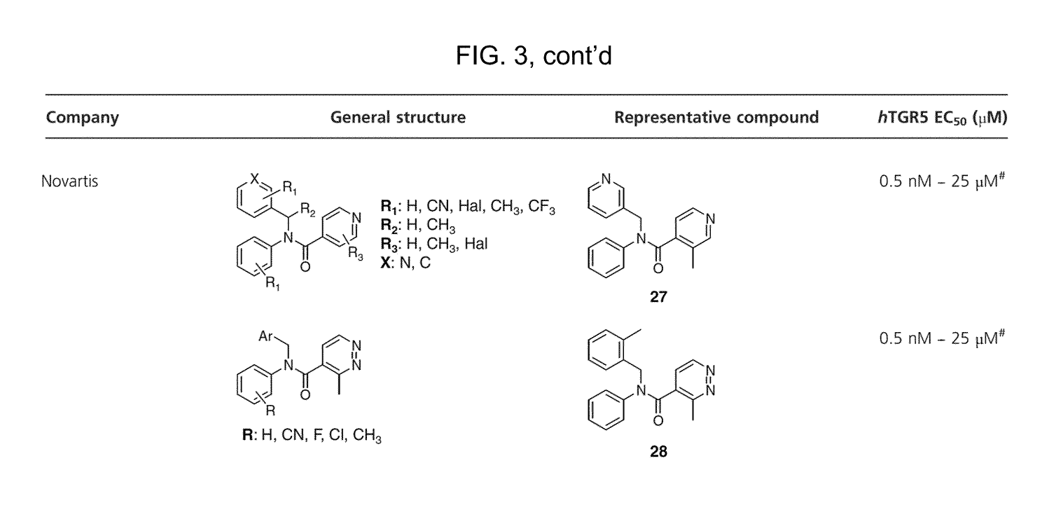

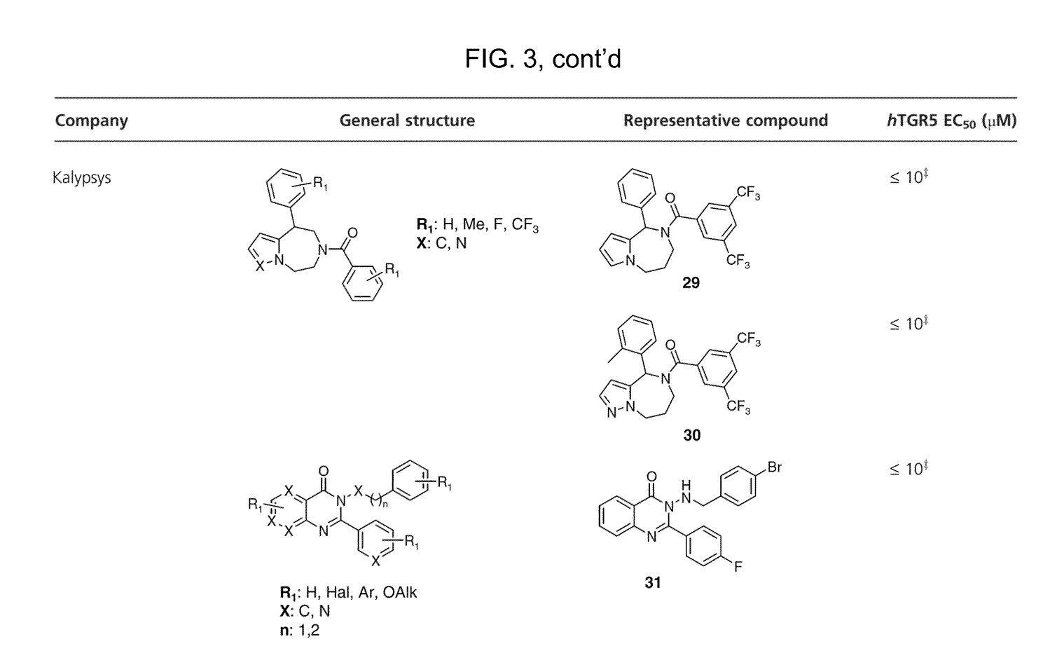

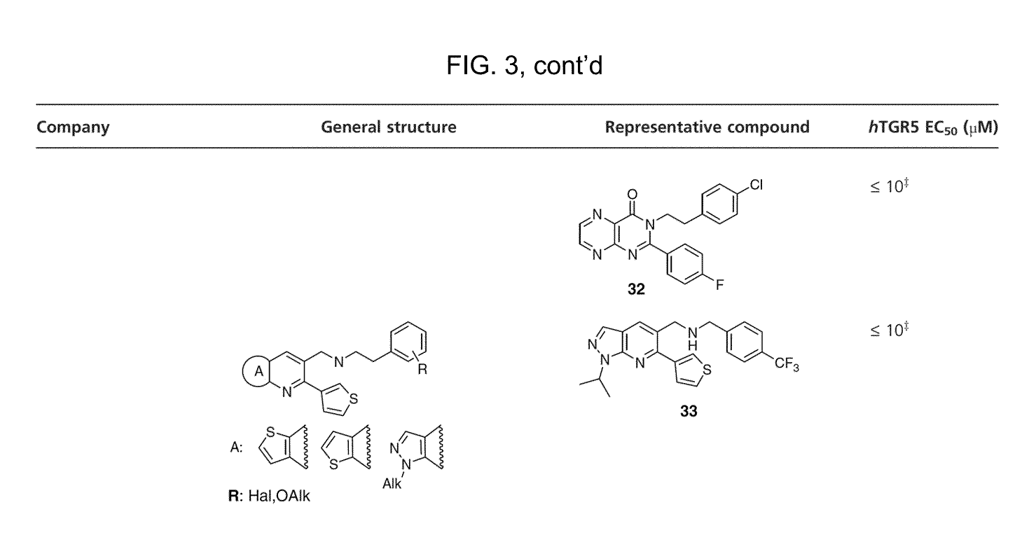

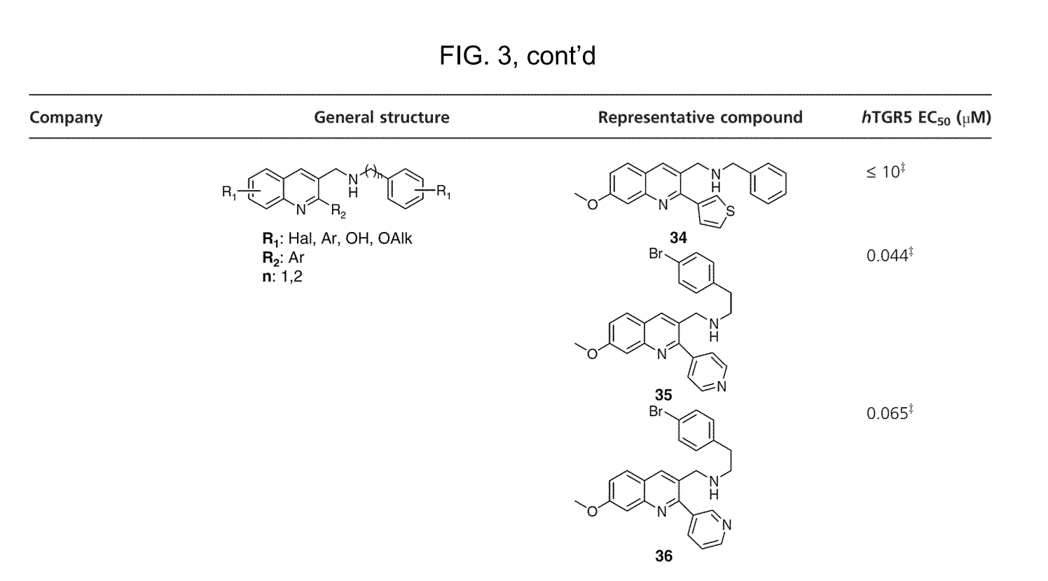

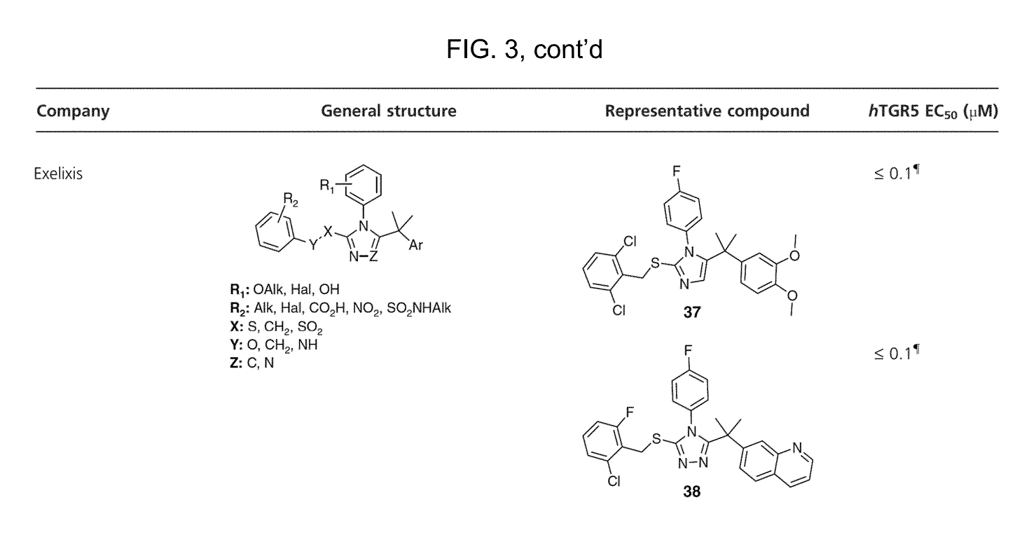

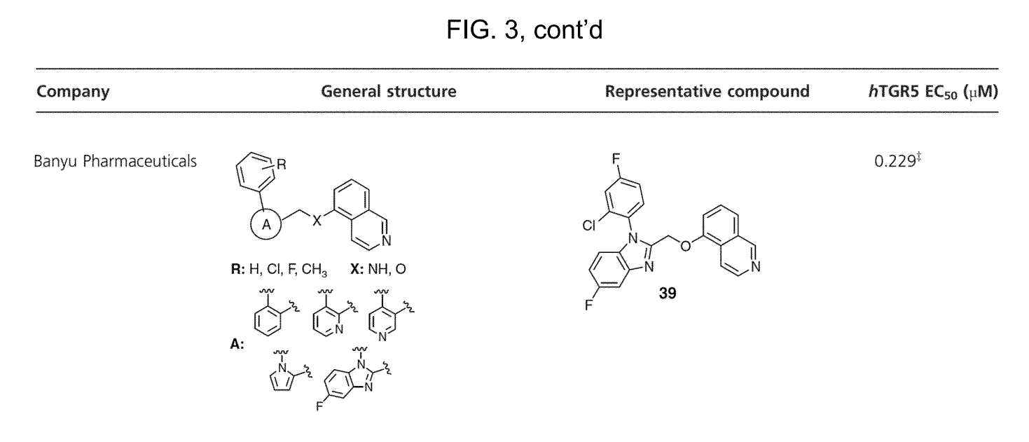

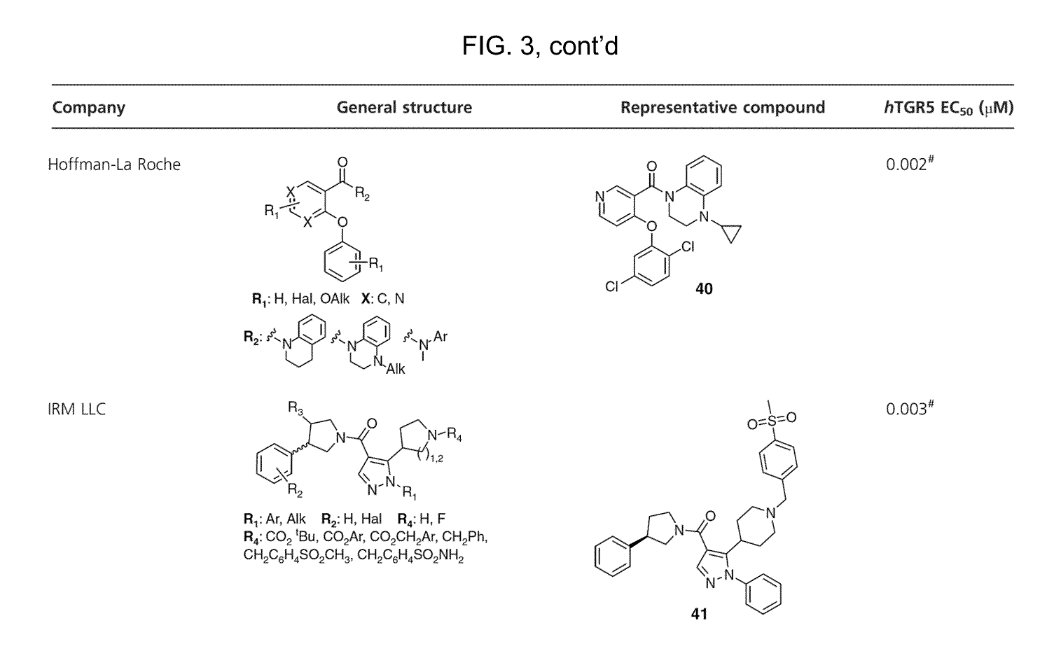

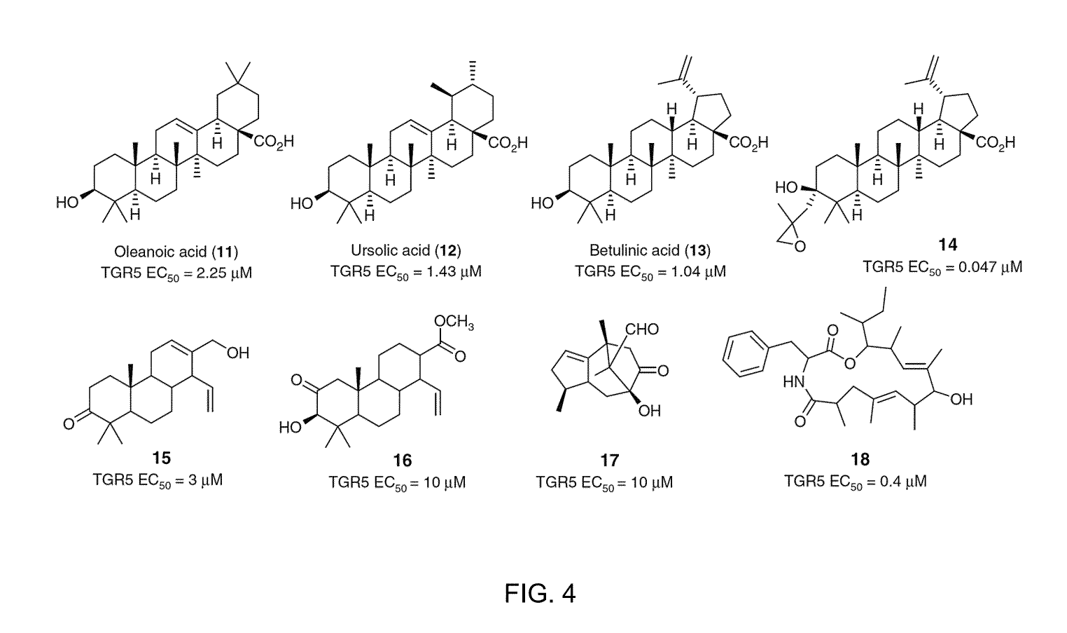

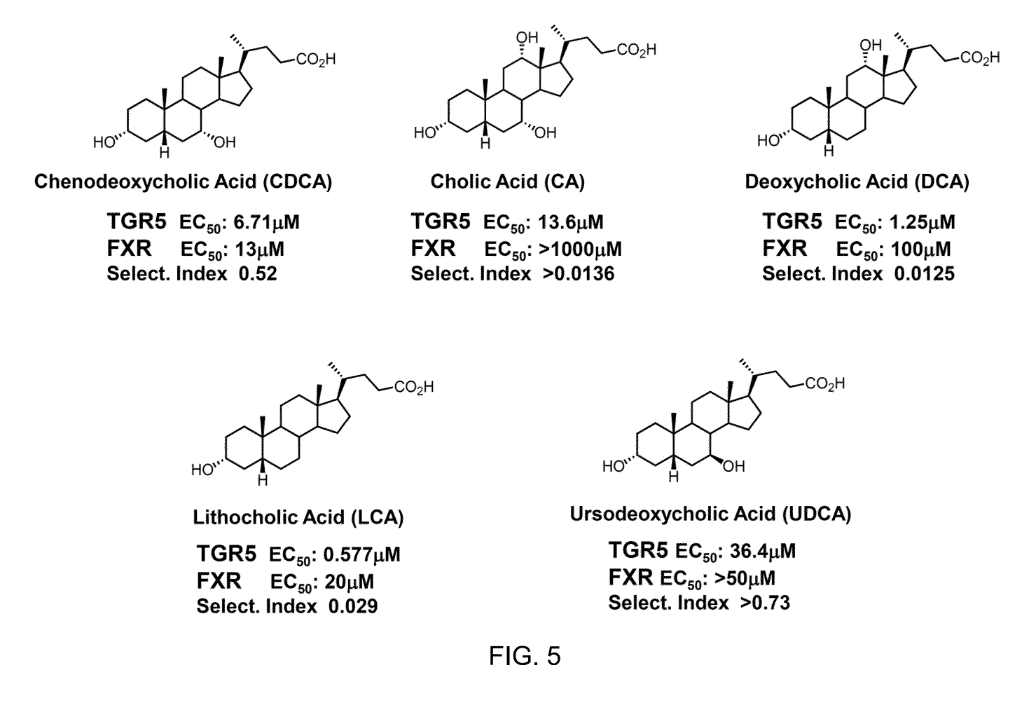

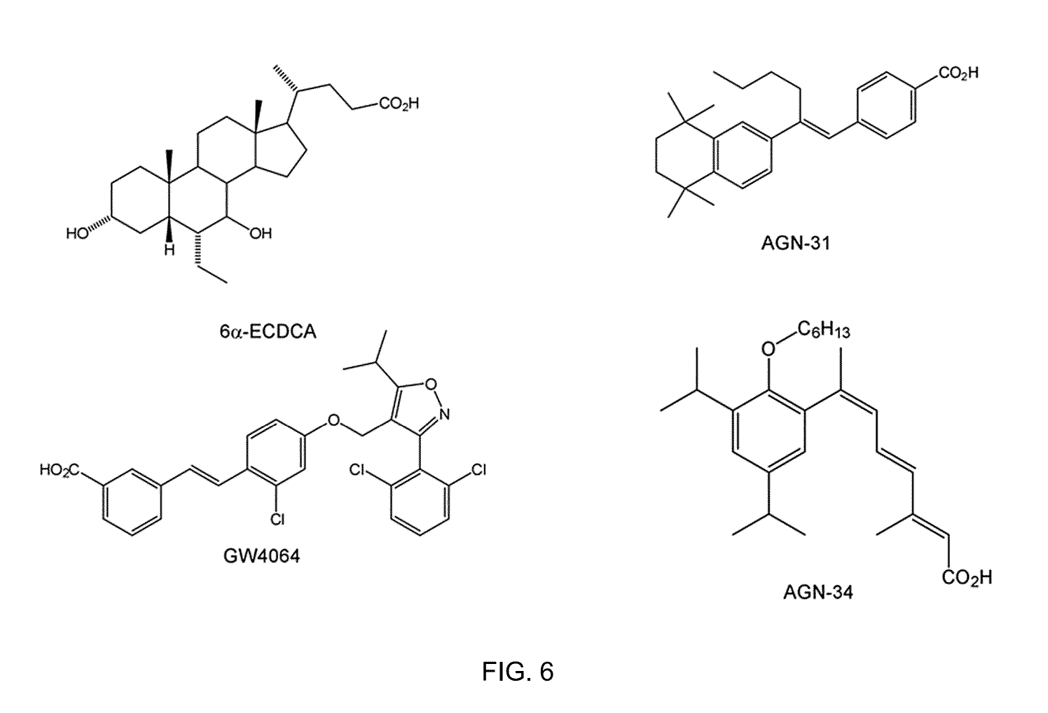

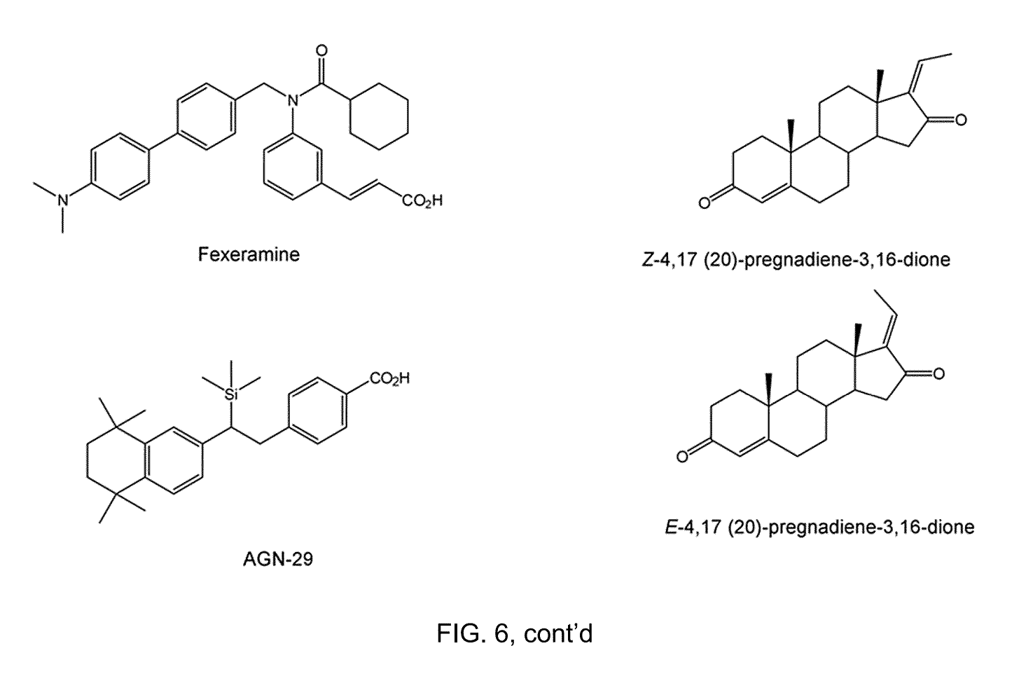

### EXAMPLE 1

Inhibitory effects of PG102 and synthetic FXR agonists like GW4064 and 6-ECDCA on pro-inflammatory cytokine secretion by THP1 cells.

### Materials and Methods:

Cells: THP1 is a human monocytic cell line derived from an acute monocytic leukemia patient (Tsuchiya S et al (1980). Int. J. Cancer 26 (2): 171-6.). The Jurkat cell line is described in Schneider U et al., (1977). Int J Cancer 19 (5): 621-6. Briefly, on day 1, THP-1 and Jurkat 39.8/50 human cells will be cultured in Iscove's Modified Dulbecco's Medium (IMDM, BioWhittaker, catalogue number BE12-722F supplemented with 10% foetal bovine serum (Gibco, ref 10270-106) and 50 μg/mL gentamycin (Invitrogen, catalogue number 15750-045). Subsequently, the THP1 cells will be left untreated or will be pretreated
with rhuIFNγ (1000 U/mL, PeproTech) for 48 h to upregulate CD40 expression
with an FXR agonist (GW4064 (Sigma G5172), or 6-ECDCA (Cayman, 11031)) for 18 h

On day 3 of the bioassay, THP-1 cells will be washed and cultured according to the following scheme:

| | | | LPS | |
|---|---|---|---|---|
| | | Jurkat 39.8/50 | (1 μg/ml, | |
| | THP1 cells | cells * | sigma) | Test sample |
| 1 | Untreated | — | X | — |
| 2 | Untreated | — | X | PG102** |
| 3 | Untreated | — | X | FXR agonist |
| 4 | Untreated | — | X | PG102 + FXR |
| | | | | agonist |
| 5 | IFNγ | X | — | — |
| | pretreated | | | |
| 6 | IFNγ | X | — | PG102 |
| | pretreated | | | |
| 7 | IFNγ | X | — | FXR agonist |
| | pretreated | | | |
| 8 | IFNγ | X | — | PG102 + FXR |
| | pretreated | | | agonist |
| 9 | IFNγ | X | X | — |
| | pretreated | | | |
| 10 | IFNγ | X | X | PG102 |
| | pretreated | | | |
| 11 | IFNγ | X | X | FXR agonist |
| | pretreated | | | |
| 12 | IFNγ | X | X | PG102 + FXR |
| | pretreated | | | agonist |
| 13 | Pretreated | — | X | — |
| | with FXR | | | |
| | agonist | | | |
| 14 | Pretreated | — | X | PG102 |
| | with FXR | | | |
| | agonist | | | |

* human T cell cell line expressing CD40L. THP1 cells and J39.8/50 cells will be cultured in a 1:1 ratio
**PG102; PanGenetics, Batch PANY001, June 2011

All conditions will be done in triplicate in round bottomed cell culture plates (Nunclon™) in the following order: 50 μL of THP-I cells (equivalent to 2×10⁴ cells per well), 50 μL of the test sample and 50 μL J39.8/50 cells. The total volume will be 150 μl per well. Cells will be incubated at 37° C. in a humidified 5% CO2 atmosphere for 48 h.

On day 5, after a culture period of 48 h, 70 μL of cell culture supernatants will be collected and transferred to a low-binding round bottomed microtitre plate. The harvested cell culture supernatants will be assayed for multiple cytokines including TNF, IL-6, IL-1β, and IL-10 using a multiplex cytokine assay (Luminex) in accordance with the manufacturer's instructions.

The percentage inhibition of cytokine secretion achieved with the test samples in the different test conditions will be calculated.

### EXAMPLE 2

Synergistic inhibitory effects of PG102 and the synthetic FXR agonist 6-ECDCA on pro-inflammatory cytokine secretion by peripheral blood mononuclear cells (PBMC).

### Materials and Methods:

PBMC were freshly isolated from heparinized human blood using Fycoll density gradient centrifugation (Histopaque; Sigma Diagnostics). PBMC were cultured in RPMI containing 10% FCS in round-bottom 96-well plates at a concentration of 5×10E5 cells/mL. Two different stimuli were used to induce cytokine secretion from PBMC:
1. PBMC were cultured in the presence of IFN-γ (250 U/mL) for 24 hours to induce upregulation of CD40. The CD40 pathway was subsequently activated for 24 hours with megaCD40L (100 ng/mL, Enzo Life Sciences).
2. PBMC were cultured in the presence of IFN-γ for 24 hours to induce upregulation of CD40. Cells were subsequently stimulated for 24 hours with megaCD40L (100 ng/mL) and LPS (100 ng/mL).

Stimulated PBMC were cultured in the absence or presence of variable concentrations of PG102 (5, 10 and 100 ng/mL) and/or 6-ECDCA (0.1, 1 and 5 μM). PG102 was added simultaneously with the stimulus. In contrast, 6-ECDCA was added 3 hours before adding the stimulus. At the end of the culture period, supernatants were collected and stored at −80° C. until cytokine analysis was performed. The BD cytometric Bead Array (CBA) Human Inflammatory Cytokines Kit (BD Biosciences) was used to measure IL-1β, IL-8, IL-6, TNF and IL-12p70 levels in the culture supernatants. The assay was performed according to the manufacturer's instructions. In brief, capture beads for the cytokines of interest were mixed with supernatant or human inflammatory cytokine standards and PE detection reagent. Samples were incubated for 3 hours at room temperature in the dark. Subsequently, samples were washed and analyzed on a FACS CANTO II cytometer (BD Biosciences). Data was analyzed using de FCAP Array software (BD Biosciences).

### Results:

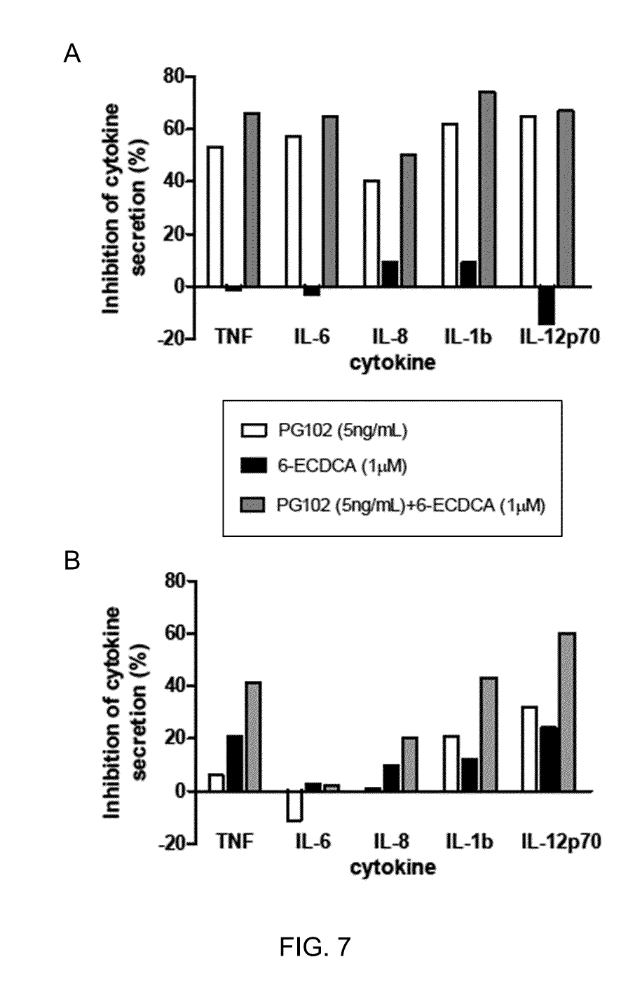

Upon stimulation of the CD40-CD154 pathway, significant amounts of TNF (733 pg/mL), IL-6 (5.3 ng/mL) and IL-8 (19.5 ng/mL) were produced. IL-12p70 (50 pg/mL) was also produced under these stimulation conditions. IL-1β (7 pg/mL) was hardly detectable. PG102 inhibited this cytokine release from PBMC in a dose-dependent fashion, with 100 ng/mL PG102 inhibiting 89%, 87%, 78%, 88%, and 93% of the TNF, IL-6, IL-8, IL-1β and IL-12p70 release, respectively. 6-ECDCA alone, used at a concentration of 0.1 or 1 μM, did not inhibit release of these cytokines. However, 5 μM of 6-ECDCA inhibited TNF, IL-6, IL-8, IL-1β and IL-12p70 release with 14%, 18%, 17%, 35% and 20%, respectively. Adding a combination of PG102 and 6-ECDCA to the cultures inhibited cytokine release more than PG102 or 6-ECDCA alone, also when using 6-ECDCA in a concentration (1 μM) that did not have an inhibitory effect on cytokine release when given in the absence of PG102. In FIG. 7A, the percentage inhibition of cytokine secretion is depicted for PG102 (5 ng/mL) alone, 6-ECDCA (1 μM) alone and the combination of PG102 (5 ng/mL) and 6-ECDCA (1 μM).

Alternatively, PBMC were stimulated through the CD40 pathway in combination with a Toll-like receptor stimulus (LPS). LPS is a major component of the outer membrane of Gram-negative bacteria and elicits strong immune responses in humans. Binding of LPS to the TLR4 receptor leads, like stimulation of the CD40 pathway, to the activation of NF-κB and the production of pro-inflammatory cytokines. Under these stimulation conditions, all evaluated cytokines were secreted in high amounts (TNF: 9 ng/mL; IL-6: 23 ng/mL; IL-8: 23 ng/mL; IL-1β: 100 pg/mL; IL-12p70: 1500 pg/mL). PG102 alone was able to dose-dependently inhibit TNF, IL-12p70 and IL-1b secretion, but IL-8 and IL-6 secretion were hardly affected. Clearly, PG102 was less potent under these stimulation conditions, compared to exclusive stimulation of the CD40 pathway. PG102 (100 ng/mL) inhibited TNF, IL-6, IL-8, IL-1β and IL-12p70 by 38%, 0%, 17%, 43% and 67%, respectively. 6-ECDCA has been shown before to inhibit LPS-induced TNF production in a dose-dependent fashion (Gadaleta R M, et al. Gut 2011; 60(4):463-72). Here, we show that 6-ECDCA is not very effective in inhibiting proinflammatory cytokine release which is induced by activation of the CD40 pathway alone or in combination with the TLR-4 pathway. 6-ECDCA (5 μM) inhibited TNF, IL-6, IL-8, IL-1β and IL-12p70 by 21%, 0%, 9%, 12% and 22%, respectively. The combination of PG102 and 6-ECDCA was more effective in inhibiting TNF, IL-6, IL-8, IL-1β and IL-12p70 release than PG102 or 6-ECDCA alone. This is shown in FIG. 7B for PG102 and 6-ECDCA used at a concentration of 5 ng/mL and 1 μM, respectively.

The data show that PG102 in combination with 6-ECDCA inhibit secretion of all proinflammatory cytokines analyzed in this study, more effectively compared to PG102 or 6-ECDCA alone. Especially, when the cytokine-inducing stimulus is stronger, the combination of 6-ECDCA and PG102 has a synergistic inhibitory effect on proinflammatory cytokine release. These data show that 6-ECDCA in combination with PG102 can block inflammation independent of the stimulus, which implicates that it is not relevant whether a microbial component or an autoimmune process is underlying the proinflammatory cytokine release. The data also show that it is possible to use lower concentrations of these agents when used together to allow for a better safety profile without loss of effectivity.

### EXAMPLE 3

Synergistic inhibitory effects of MR-1 and the synthetic FXR agonists 6-ECDCA in the DSS-induced colitis mouse model.

### Materials and Methods:

Colitis was induced in C57/Bl6 wild type mice by administration of 2.5% (wt/vol) Dextran Sodium Sulphate (DSS; MW. 36000-50000 Da, MP Biochemicals Inc) in drinking water for 8 days. Pharmacological activation of FXR was accomplished by treatment with 6-ethyl-chenodeoxycholic acid (6-ECDCA). 6-ECDCA (10 mg/kg/day) or vehicle were administered by oral gavage for three days prior to the start of DSS-treatment, and continued until the end of the DSS-treatment. At the second day of DSS treatment (day 4 of 6-ECDCA treatment), mice were given an intraperitoneal (i.p.) injection with 250 μg of the hamster antibody against mouse CD40L, MR-1, or control IgG (LEAF™ Purified Armenian Hamster IgG Isotype Ctrl Antibody, Biolegend).

There were 5 treatment groups (n=10 mice/group) in the experiment:

1. −DSS, +vehicle by orale gavage (o.g.)+controle IgG i.p.
2. +DSS, +vehicle o.g.+control IgG i.p.
3. +DSS, +vehicle o.g.+anti-CD40L i.p.
4. +DSS, +6-ECDCA o.g.+control IgG i.p.
5. +DSS, +6-ECDCA o.g.+anti-CD40L i.p.

Daily changes in body weight were assessed and the body weight at day 2-11 was expressed relative to the body weight at day 1. For intestinal permeability assays, mice were given FITC by oral gavage after 8-days of DSS-treatment. 4 h after FITC administration, mice were sacrificed and the amount of fluorescence in the blood was determined as a marker for permeability. The colon was isolated and colon length was measured. Spleens were collected and cells were isolated. Cells were stained with an antibody mixture to determine the composition of the immune cells in spleen by FACS analysis. Granulocytes were identified based on GR-1 and CD11b expression and expressed as percentage of living cells in the spleen. Finally, spleen cells were stimulated in vitro for 4.5 h with PMA and ionomycin. Culture supernatants were collected and TNF was measured using an ELISA.

### Results:

The FXR receptor agonist 6-ECDCA has been shown before to interfere with chemically induced intestinal inflammation, with improvement of colitis symptoms, inhibition of epithelial permeability, and reduced goblet cell loss (Gadaleta R M, et al. Gut 2011; 60(4):463-72). MR-1, an antagonistic anti-CD40L antibody, was effective in an experimental colitis model in SCID mice reconstituted with syngeneic CD45RBhighCD4+ T cells (Liu Z, et al. J. Immunol 2000; 164(11):6005-11). In the present study, the dosing scheme of 6-ECDCA was identical to that reported by Gadaleta et al. Indeed, also in the present study 6-ECDCA interfered with the colitis disease process induced by DSS. MR-1, in contrast, was dosed suboptimally (given only once, one day after colitis induction) to allow for synergistic effects of the combination of CD40 pathway blockade and FXR receptor activation. Lowering the dose and frequency of CD40 pathway blockade in the clinic, lowers the risk of side effects and unwanted immune suppression. Results from the present study show that the applied dosing scheme of MR-1, when given alone, was not sufficient to interfere with the colitis disease process, but when given in combination with FXR receptor agonist 6-ECDCA is able to interfere with the colitis disease process.

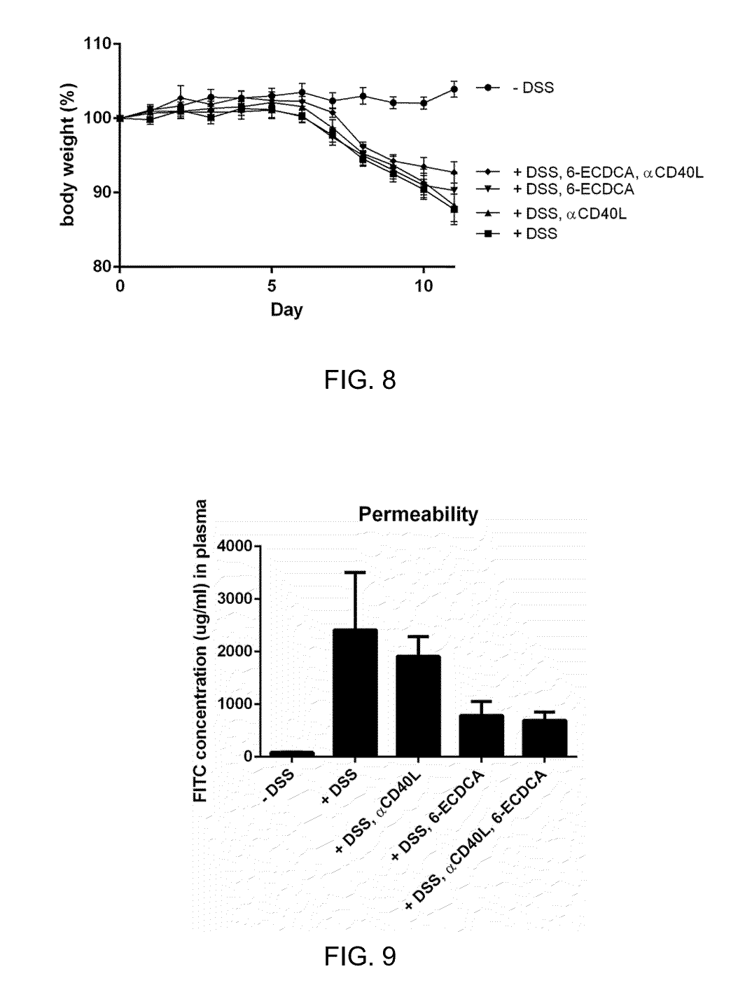

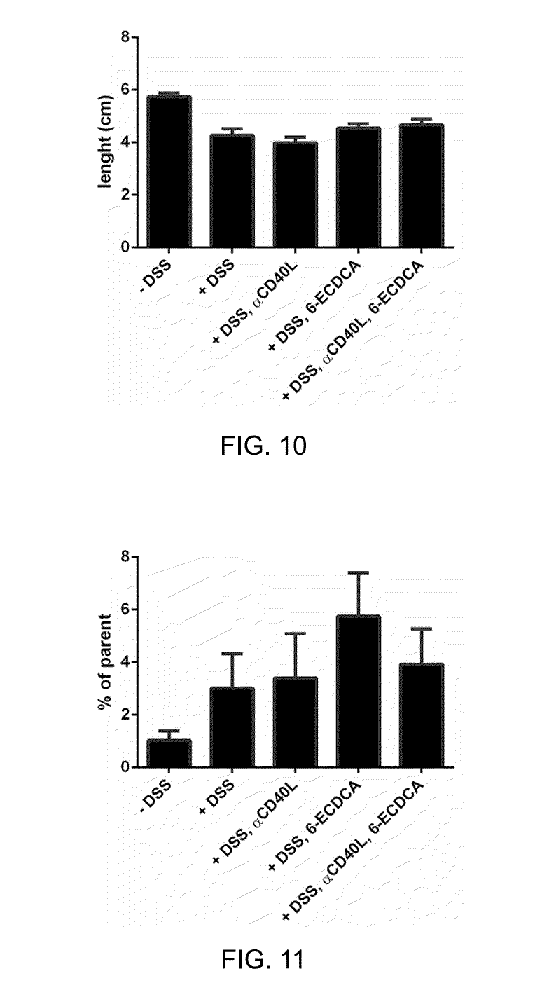

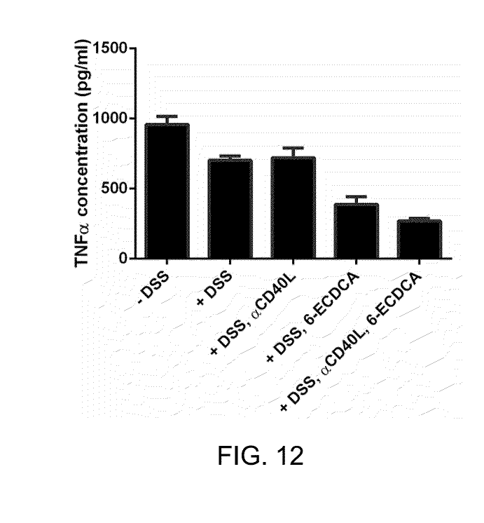

As expected, DSS caused a drop in the body weight starting at day 7, 4 days after the start of the DSS administration. This drop in body weight was least in group 5, mice receiving both 6-ECDCA and MR-1 (FIG. 8). Also, of the mice receiving DSS, intestinal permeability was least impaired in the combination treatment group (FIG. 9). Colon shortening is a hallmark of inflammation. DSS causes mainly inflammation, and thus shortening, of the colon. The length of the colon of the mice receiving the combination DSS+6-ECDCA+αCD40L is not significantly different from the length of the colon of the mice which did not receive DSS (FIG. 10). 6-ECDCA appeared to cause an increase in granulocytes in the spleen, an effect which was reduced by combining 6-ECDCA with αCD40L (FIG. 11). FIG. 12 shows that spleen cells isolated from mice receiving both 6-ECDCA and αCD40L produced less TNF upon in vitro stimulation compared to spleen cells isolated from mice from the other DSS-treated groups. (group 2-4) Altogether, whereas MR-1 did not have an effect, combining MR-1 and 6-ECDCA had superior effects compared to 6-ECDCA alone in this mice colitis model on multiple outcome measures. Hence, when FXR-receptor activation and anti-CD40 blockade are applied in autoimmune disease of the gastrointestinal tract including the liver, the combination will allow to use a milder dosing scheme with lower concentrations of each agent used and a lower dosing frequency. The combination of FXR receptor activation and CD40 blockade contributes to an improved safety profile and more effective inhibition of inflammation.

### CITED ART

1. Ranheim E A, Kipps T J. Activated T cells induce expression of B7/BB1 on normal or leukemic B cells through a CD40-dependent signal. J Exp Med (1993); 177: 925-35.
2. Hasbold J, Johnson-Leger C, Atkins C J, Clark E A, Klaus G G B. Properties of mouse CD40: cellular distribution of CD40 and B cell activation by monoclonal anti-mouse CD40 antibodies. Eur J Immunol (1994); 24: 1835-42.
3. Alderson M R, Armitage R J, Tough T W, Strockbine L, Fanslow W C, Spriggs M K. CD40 expression by monocytes: regulation by cytokines and activation of monocytes by the ligand for CD40. J Exp Med (1993); 178: 669-74.
4. Kiener P A, Moran-Davis P, Rankin B M, Wahl A F, Aruffo A, Hollenbaugh D. Stimulation of CD40 with purified soluble gp39 induces proinflammatory responses in human monocytes. J Immunol (1995); 155: 4917-25.
5. Shu U, Kiniwa M, Wu C Y, et al. Activated T cells induce interleukin-12 production by monocytes via CD40-CD40 ligand interaction. Eur J Immunol (1995); 25: 1125-8.
6. Cella M, Scheidegger D, Palmer-Lehmann K, Lane P, Lanzavecchia A, Alber G. Ligation of CD40 on dendritic cells triggers production of high levels of interleukin-12 and enhances T cell stimulatory capacity: T-T help via APC activation. J Exp Med (1996); 184: 747-52.
7. van Kooten C, Banchereau J. Functions of CD40 on B cells, dendritic cells and other cells. Curr Opin Immunol (1997); 9: 330-7.
8. Cayabyab M, Phillips J H, Lanier L L. CD40 preferentially costimulates activation of CD4+ T lymphocytes. J Immunol (1994); 152: 1523-31.
9. Hermann P, Van-Kooten C, Gaillard C, Banchereau J, Blanchard D. CD40 ligand-positive CD8+ T cell clones allow B cell growth and differentiation. Eur J Immunol (1995); 25: 2972-7.
10. Grewal I S, Flavell R A. CD40 and CD154 in cell-mediated immunity. Annu Rev Immunol (1998); 16: 111-35.
11. Henn V, Slupsky J R, Grafe M, et al. CD40 ligand on activated platelets triggers an inflammatory reaction of endothelial cells. Nature (1998); 391: 591-4.
12. Kawai T, Andrews D, Colvin R B, Sachs D H, Cosimi A B. Thromboembolic complications after treatment with monoclonal antibody against CD40 ligand. Nat Med (2000); 6: 114.
13. Knosalla C, Gollackner B, Cooper D K. Anti-CD154 monoclonal antibody and thromboembolism revisited. Transplantation (2002); 74: 416-17.
14. de Boer M, Conroy J, Min H Y, Kwekkeboom J. Generation of monoclonal antibodies to human lymphocyte cell surface antigens using insect cells expressing recombinant proteins. J Immunol Meth (1992); 152: 15-23.
15. Kwekkeboom J, de Rijk D, Kasran A, Barcy S, de Groot C, de Boer M. Helper effector function of human T cells stimulated by anti-CD3 Mab can be enhanced by co-stimulatory signals and is partially dependent on CD40-CD40 ligand interaction. Eur J Immunol (1994); 24: 508-17.
16. Laman J D, 't Hart B A, Brok, H P M, et al. Protection of marmoset monkeys against EAE by treatment with a murine antibody blocking CD40 (mu5D12). Eur J Immunol (2002); 32: 2218-28.
17. Boon L, Laman J D, Ortiz-Buijsse A, et al. Preclinical assessment of anti-CD40 Mab 5D12 in cynomolgus monkeys. Toxicology (2002); 174: 53-65.

TABLE 1
Various patented TGR5 agonists (from Gioiello et al (2012) Expert Opin. Ther. Patents. Vol 22: pp 1399-1414).

| Classification | | Chemical class (Compound number) | Applicant | Publication number |
|---|---|---|---|---|
| Steroidal compounds | | BAs (7) | Intercept Pharmaceuticals | WO091540A2 |
| | | Bile sulfates (8) | | WO002573A2 |
| | | Bile sulfonates (9) | | US0172198A1 |
| | | BAs (10) | | WO053859A1 |
| | | Lithocholic amides | Novartis | WO009407A2 |
| Non-steroidal | Natural-derived | Cleistanthanes (15, 16) | Merck | WO146772A1 |
| compounds | compounds | Patchoulenes (17) | | WO146772A1 |
| | | Pentadecanolides (18) | | WO146772A1 |
| | Compounds from | Tetrahydropyrimidines (19) | Takeda Pharmaceuticals | WO043468A1 |
| | chemical libraries | Oxazepines (20) | | EP1591120A1 |
| | | Oxazepines (21) | | US136778 |
| | | Thiazol-4-carboxamides (22) | Arena Pharmaceuticals | WO116653A2 |
| | | Bis-Sulfonamides (23) | SmithKline Beecham Corp. | WO127505A2 |
| | | Heterocyclic amides (27, 28) | Novartis | WO110237A2 |
| | | | | WO125627A1 |
| | | Diazepines (29, 30) | Kalypsys | WO06722A1 |
| | | Quinazolines (31) | | WO067219A2 |
| | | Pteridines (32) | | WO014739A2 |
| | | Pyridines (33) | | WO016846A1 |
| | | Quinolines (34) | | WO097976A1 |
| | | Imidazoles (37) | Exelixis | WO093845A1 |
| | | Triazoles (38) | | |
| | | Isoquinolines (39) | Banyu Pharmaceuticals | WO117084A1 |
| | | | | WO117090A1 |
| | | Aryl amides (40) | Hoffmann-La Roche | WO049302A1 |
| | | | | WO089099A1 |
| | | Pyrazoles (41) | IRM LCC | WO082947A1 |

BA—Bite acid

TABLE 2
various TGR5 agonists (from Gioiello et al (2012) Expert Opin. Ther.
Patents. Vol 22: pp 1399-1414).

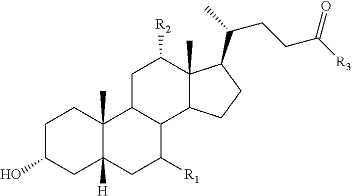

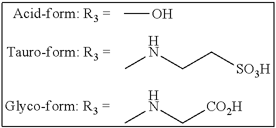

| | | | Acid-form | Tauro-form | Glyco-form |
|---|---|---|---|---|---|
| Trivial name | R₁ | R₂ | EC₅₀ (μM) | EC₅₀ (μM) | EC₅₀ (μM) |
| LCA (1) | —H | —H | 0.58 | 0.29 | 0.54 |
| DCA (2) | —H | —OH | 1.25 | 0.79 | 1.18 |
| CDCA (3) | α-OH | —H | 6.71 | 1.92 | 3.88 |
| CA (4) | α-H | —OH | 13.6 | 4.95 | 13.6 |

BA: Bile acid;
CA: Cholic acid;
CDCA: Chenodeoxycholic acid;
DCA: Deoxycholic acid;
LCA: Lithocholic acid.

1. A CD40 signalling inhibitor and a further compound for use in the treatment of chronic inflammatory or autoimmune disease in an individual in need thereof, wherein the further compound is a bile acid, a bile acid derivative, an TGR5-receptor agonist, an FXR-receptor agonist or a combination thereof.

2. A CD40 signalling inhibitor and a further compound for use according to claim 1, wherein said chronic inflammatory or autoimmune disease is a chronic inflammatory or autoimmune disease of the liver, of the kidney, of the gastrointestinal tract, of the cardiovascular system or the metabolic system.

3. A CD40 signalling inhibitor and a further compound for use according to claim 1, wherein said chronic inflammatory or autoimmune disease of the liver is, primary biliary cirrhosis (PBC), bile acid diarrhea (chronic diarrhea), primary sclerosing cholangitis (PSC), autoimmune hepatitis, liver transplant associated graft versus host disease, portal hypertension, non-alcoholic steatohepatitis (NASH) or non-alcoholic fatty liver disease (NAFLD).

4. A CD40 signalling inhibitor and a further compound for use according to claim 1, wherein said chronic inflammatory or autoimmune gastrointestinal disease is a chronic inflammation of the pancreas, Crohn's disease, or ulcerative colitis.

5. A CD40 signalling inhibitor and a further compound for use according to claim 1, wherein said chronic inflammatory cardiovascular disease is atherosclerosis.

6. A CD40 signalling inhibitor and a further compound for use according to claim 1, wherein said chronic inflammatory or autoimmune disease of the metabolic system is obesity, insulin resistance, metabolic syndrome, type I diabetes or type II diabetes.

7. A CD40 signalling inhibitor and a further compound for use in the prevention of cancer and/or fibrosis, wherein the further compound is a bile acid, a bile acid derivative, an TGR5-receptor agonist, an FXR-receptor agonist or a combination thereof.

8. A CD40 signalling inhibitor and a further compound for use according to claim 1, wherein said bile acid or bile acid derivative is an FXR and/or TGR5 signalling activator (agonist).

9. A CD40 signalling inhibitor and a further compound for use according to claim 1, wherein said bile acid derivative comprises a chenodeoxycholic acid derivative, preferably 6-alpha-ethyl chenodeoxycholic acid, or a 23-substituted bile acid.

10. A CD40 signalling inhibitor and a further compound for use according to claim 1, wherein said bile acid is ursodeoxycholic acid or chenodeoxycholic acid.

11. A CD40 signalling inhibitor and a further compound for use according to claim 1, wherein said CD40 signalling inhibitor comprises an antibody that binds CD40, or a fragment or derivative thereof.

12. A CD40 signalling inhibitor and a further compound for use according to claim 11, wherein said antibody that binds CD40, comprises the variable region of antibody 5D12, ch5D12, PG102, CHIR-12.12, ASKP1240, or derivative thereof.

13. A method for the treatment of an individual suffering from a chronic inflammation, said method comprising administering to the individual in need thereof, an CD40 signalling inhibitor and a further compound according to claim 1.

14. A kit comprising a CD40 signalling inhibitor and a further compound wherein the further compound is a bile acid, a bile acid derivative, an TGR5-receptor agonist, an FXR-receptor agonist or a combination thereof.

15. A kit according to claim 14, for use in the treatment of chronic inflammatory or autoimmune disease in an individual in need thereof or for use in the prevention of cancer and/or fibrosis in an individual in need thereof.
